(19) United States
(12) Patent Application Publication
(10) Pub. No.: US 2006/0008498 A1
(43) Pub. Date: Jan. 12, 2006

(54) Nano-particle production

(76) Inventor: Tzer-Fen D. Chen, McLean, VA (US)

Correspondence Address: FISH & RICHARDSON PC, P.O. BOX 1022, MINNEAPOLIS, MN, 55440-1022, US

(21) Appl. No.: 10/889,615

(22) Filed: Jul. 12, 2004

(51) Int. Cl. A61F 13/00; A61K 9/14

(52) U.S. Cl. 424/422; 424/489

(57) ABSTRACT

Larger particles (e.g., larger than 100 μm) of a material are processed to generate smaller particles having dimensions smaller than 200 nm. At least a portion of the processing is performed under a cryogenic condition and based on at least a physical property of the material under the cryogenic condition.

### TECHNICAL FIELD

This description relates to the production of nano-particles.

### BACKGROUND

The particle size of a compound often affects its physical and chemical properties, such as apparent solubility, color, wetting, suspension stability, compaction behavior, appearance, and tactility. These properties are important in many areas. For example, in the field of pharmaceutics, drug delivery, drug bioavailability, production processes, product properties, product stability, and physiological compatibility are closely related to the particle sizes of pharmaceutical compounds. When their particle sizes are reduced to smaller than 200 nano-meters (nm), many compounds display useful properties that are quite different from those of larger particles of the same compound. Nano-particles can be produced by, for example, expansion of a supercritical fluid and by supercritical fluid anti-solvent precipitation.

### DESCRIPTION OF DRAWINGS

FIG. 1 shows a nano-particle generator.

FIG. 2 shows a powder feeder.

FIG. 3 shows a solid atomizer.

FIGS. 4 and 5 show cryogenic gas generators.

FIG. 6 shows a cryogenic jet mill.

FIG. 7 shows a cyclone collector.

### SUMMARY OF INVENTION

In general, in one aspect, the invention features a method including processing larger particles of a material to generate smaller particles having at least one dimension smaller than 200 nm, at least a portion of the processing performed under a cryogenic condition and based on at least a physical property of the material under the cryogenic condition.

Implementations of the invention may include one or more of the following features.

The cryogenic condition includes a condition in which the temperature is less than −40° C. A physical property includes a greater tendency to crack under the cryogenic condition. The larger particles initially have at least one dimension larger than 100 μm. At least 5 percent of the larger particles are processed into smaller particles having at least one dimension smaller than 200 nm. At least 5 percent of the larger particles are processed into smaller particles having three dimensions smaller than 200 nm.

The processing includes milling the larger particles. The milling includes using a pressurized cryogenic gas to cause the particles to grind against or collide with one another. The cryogenic gas includes at least one of air, nitrogen, and inert gas. The inert gas includes helium.

The processing includes at least one treatment in addition to the milling, the additional treatment based on at least a physical property of the material under the cryogenic condition. The additional treatment includes imposing ultrasonic waves to the particles. The additional treatment includes imposing microwaves to the particles.

A physical property includes the material forming cracks when subject to ultrasonic vibrations under the cryogenic condition. The processing includes imposing ultrasonic waves on the particles. The material has a physical property such that the material forms cracks when the temperature of the material rises at least 20° C. per second from a first temperature under the cryogenic condition to a second temperature. The processing includes using microwaves to increase the temperature of the particles. The processing includes exposing the particles alternately to a cryogenic condition and a non-cryogenic condition.

The method includes generating a cryogenic gas to process the particles. Generating the cryogenic gas includes passing a gas through a passage that is cooled by liquid nitrogen, or passing a gas through liquid nitrogen. The passage includes a coil tube immersed in the liquid nitrogen. The gas includes at least one of air, nitrogen, and an inert gas. The inert gas includes helium. The cryogenic gas has a pressure at least 100 psi.

The method includes spraying a liquid from a higher pressure area to a lower pressure area to generate the larger particles. The method includes cooling the lower pressure area with cryogenic gas. The method includes generating the liquid by dissolving a material in a solvent. The solvent includes at least one of acetone, chloroform, alcohol, ether, petroleum ether, benzene, and water. The alcohol includes at least one of methanol, ethanol, and isopropyl alcohol. The material includes plastic.

The larger sized particles include herbs, calcium oxalate, calcium sulfate, calcium phosphate, silicon dioxide, cellulose and herbs, insulin, taxine, griseofulvin, albuterol sulfate, ibuprofene, lecithin, plastic, vitamins, iron oxide, or paclitaxcel. The material is in a solid state or a liquid state at 20° C.

In general, in another aspect, the invention features a method that includes generating a cryogenic gas by passing a gas through a passage that is cooled by liquid having a temperature less than or equal to −40° C.

Implementations of the invention may include one or more of the following features.

The liquid has a temperature less than or equal to −100° C. The liquid includes liquid nitrogen. The passage includes a coil tube immersed in the liquid. The gas includes at least one of air, nitrogen, and an inert gas. The inert gas includes helium.

In general, in another aspect, the invention features a method that includes generating a cryogenic gas by passing a gas through liquid having a temperature less than or equal to −40° C.

Implementations of the invention may include one or more of the following features.

The liquid has a temperature less than or equal to −100° C. The liquid includes liquid nitrogen. The method includes removing oxygen in the gas by liquifying the oxygen in the liquid nitrogen. The gas includes at least one of air, nitrogen, and an inert gas. The inert gas includes helium.

In general, in another aspect, the invention features a method that includes generating small particles having three dimensions smaller than 200 nm, mixing the small particles with a liquid to generate a solution, and administering the solution to a human body through an intravenous injection.

Implementations of the invention may include one or more of the following features.

The small particles are generated according to a process in which at least a portion of the process is performed at a temperature less than or equal to −40° C. The process includes jet milling larger particles to generate the small particles. The small particles include a pharmaceutical agent.

In general, in another aspect, the invention features a method that includes generating droplets of a liquid containing a material that is dispersed in a dispersion medium, and cooling the droplets under a cryogenic condition to generate solid dispersion particles that contain the material.

Implementations of the invention may include one or more of the following features.

The cryogenic condition includes a condition in which a temperature is less than or equal to −40° C. Generating the droplets includes passing the liquid from a higher pressure region through an opening to a lower pressure region. Cooling the droplets under the cryogenic condition includes using a cryogenic gas to cool the droplets. At least a portion of the solid dispersion particles each contains a single molecule of the material.

In general, in another aspect, the invention features an apparatus that includes a jet mill to receive larger sized particles and a cryogenic gas to cause the larger sized particles to be milled into smaller sized particles, at least 5 percent of the smaller sized particles having three dimensions smaller than 200 nm.

Implementations of the invention may include one or more of the following features.

The apparatus includes a cryogenic gas generator to generate the cryogenic gas. The jet mill includes an insulated chamber to maintain a temperature equal to or below −40° C. The cryogenic gas has a temperature below or equal to −40° C.

The apparatus includes a collecting chamber to collect the smaller sized particles. The collecting chamber includes a low-pressure chamber having a pressure lower than 1 atm.

The apparatus includes an ultrasonic wave generator to direct ultrasonic waves toward the particles.

The apparatus includes a solid atomizer to generate the larger sized particles from the liquid. The solid atomizer includes a spray head to spray the liquid from a high pressure region to a low pressure region. The apparatus includes nozzles to inject cryogenic gas around the spray head to cool liquid droplets sprayed out of the spray head.

The apparatus includes a heater to heat the particles while the particles are being milled. The heater includes a microwave generator.

In general, in another aspect, the invention features an apparatus that includes a particle processor to receive larger particles and to process the larger particles into smaller particles having at least one dimension smaller than 200 nm, at least a portion of the particle processor being maintained under a cryogenic condition so that at least a portion of the processing of the larger particles is performed under the cryogenic condition.

Implementations of the invention may include one or more of the following features. The particle processor includes a jet mill to mill the larger particles under the cryogenic condition. The cryogenic condition includes a condition in which the temperature is less than or equal to −40° C.

In general, in another aspect, the invention features an apparatus that includes a container that contains a liquid maintained at a temperature less than or equal to −40° C., and a passage having at least a portion that is immersed in the liquid, the passage having a first opening to receive a gas having a temperature higher than −40° C., the passage having a second opening to output the gas after the gas passes the portion that is immersed in the liquid.

Implementations of the invention may include one or more of the following features. The liquid includes liquid nitrogen. The apparatus includes a source to generate the gas. The gas includes at least one of nitrogen, air, and an inert gas. The passage includes a coil tube.

In general, in another aspect, the invention features an apparatus that includes a container that is partially filled with a liquid maintained at a temperature less than or equal to −40° C.; a first passage having a first opening to receive a gas having a temperature higher than −40° C., the first passage having a second opening that is immersed in the liquid to output the gas into the liquid, the gas forming bubbles in the liquid that emerge from the liquid and enter a space in the container above the liquid; and a second passage to receive the gas in the open space.

Implementations of the invention may include one or more of the following features. The liquid includes liquid nitrogen. The apparatus includes a source to generate the gas. The gas includes at least one of nitrogen, air, and an inert gas.

In general, in another aspect, the invention features an apparatus that includes means for reducing the temperature of particles to a cryogenic condition, and means for processing the particles to generate smaller particles.

Implementations of the invention may include one or more of the following features. The means for reducing the temperature of particles includes a cryogenic gas generator to generate a gas having a temperature that is less than −40° C. The means for processing the particles includes a jet mill.

An advantage of using the invention to reduce the particle size of a material is that the solubility of the material can be increased. Increasing the solubility of a drug can enhance its bioavailability. Another advantage of using nano-particles is that, for some drugs, the nano-sized solid particles can be inhaled directly into the lung. The nano-sized particles can then pass through the membranes of the lung cells and enter the blood stream.

### DETAILED DESCRIPTION

Nano-particles having dimensions smaller than 200 nm can be generated by processing a material under cryogenic conditions in which the temperature is equal to or lower than −40° C. Many materials become brittle when cooled to a temperature below −40° C. To form the nano-particles, larger particles (e.g., having all three dimensions greater than 1 μm) of a material are blown into a jet mill by pressurized cryogenic gas (e.g., nitrogen at a temperature below −40° C.) to cause the larger particles to collide with one another and to grind against the inner walls of the jet mill. Because of the brittleness of the larger particles at low temperature, the collision and the grinding (also called fluidized milling) produce particles having all three dimensions smaller than 200 nm.

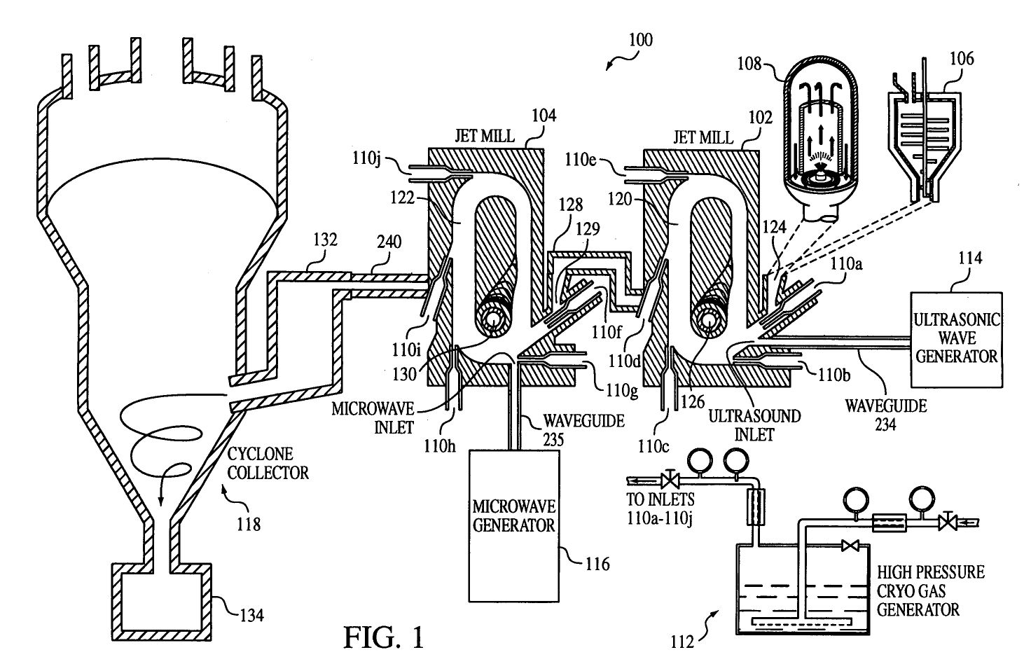

Referring to FIG. 1, a nano-particle generator 100 includes a cascaded pair of jet mills 102 and 104 to generate nano-particles having dimensions smaller than 200 nm. One or both of the jet mills 102 and 104 process particles under cryogenic conditions. By using two jet mills, one cascaded after the other, the processed particles have smaller sizes, and the sizes of the particles have a more uniform distribution. The use of two separately controlled jet mills allows the particles to be processed under different conditions within a brief span of time, such as at different temperatures and pressure conditions. Pressurized cryogenic gas, generated by a high pressure cryogenic gas generator 112, is injected through nozzles 110a-110e into the jet mill 102 to cause the particles to collide with one another and grind against the chamber walls to produce smaller particles. The gas injected into the second jet mill 104 can be cryogenic (e.g., equal to or lower than −40° C., similar to jet mill 102) or have a higher temperature.

The particles in the jet mills 102 and 104 can be treated under cryogenic conditions in different ways depending on the physical properties of the particles. Some materials are susceptible to cracking when bombarded with ultrasonic waves that have frequencies higher than 20 kHz. Some materials have high thermal expansion coefficients and are susceptible to cracking when subjected to abrupt temperature changes. When processing materials that are susceptible to ultrasonic vibrations, an ultrasonic wave generator 114 is turned on to generate ultrasonic waves that are directed toward the particles in the jet mill 102. When processing materials that have large thermal expansion coefficients, a microwave generator 116 is turned on to generate microwaves to heat the particles rapidly, causing cracks or fissures due to rapid thermal expansion. The cracks or fissures caused by the ultrasonic waves or microwaves facilitate the breaking of particles during the jet milling process. Depending on the physical properties of the material being processed, either one or both, or none, of the ultrasonic wave generator 114 and the microwave generator 116 is used.

The particles processed in the jet mill 102 are received from a powder feeder 106 or a solid atomizer 108. The powder feeder 106 is used when a material can be easily ground into nano-size from its powder form. The powder feeder 106 receives the material as a powder, and stirs the powder to prevent the small particles in the power from sticking to one another and to prevent formation of aggregates.

The solid atomizer 108 is used when the material can be intermixed with a liquid (or dissolved by a solvent) to form a liquid solution. The liquid solution is sprayed from a nozzle head into a chamber cooled by a cryogenic gas, causing the liquid to solidify and form solid dispersion particles. The process of forming solid dispersion particles will be described later. The cryogenic gas carries the solid particles into the jet mill 102.

The particles that have been processed by the jet mills 102 and 104 are forwarded to a cyclone collector 118, which has a low pressure chamber that allows water or solvent to evaporate and allows the nano-particles to fall into a collector 134 due to gravity.

The frequency of the sound waves generated by the ultrasonic wave generator 114 is adjustable. The frequency of the wave is selected based on the material to be processed. Different materials crack or fissure at different frequencies. The ultrasonic wave is sent to the chamber 120 through a waveguide 234. More than one ultrasonic wave generator may be used.

Examples of materials that are susceptible to ultrasonic vibrations include calcium oxalate, calcium sulfate, calcium phosphate, and silicon dioxide.

The microwave generator 116 has an adjustable output power. The microwave generator 116 can be turned on continuously to quickly heat the particles (e.g., using a power so that the temperature of the particles increase at least 20° C. per second), which enter the grinding chamber 122 of the jet mill 104. When the particles leave the grinding chamber 120 of the jet mill 102, the particles have low temperatures (because the particles are cooled by the cryogenic gas). When the particles enter the grinding chamber of the jet mill 104, the sudden increase in temperature may cause the particles to crack.

In one example, cryogenic gas is injected to the grinding chamber 122 of the jet mill 104 at ports 110f to 110j so that the grinding chamber 122 is maintained at a cryogenic temperature. The microwaves from the microwave generator 106 pass through a waveguide 235 and are directed to a feed port 129 of the grinding chamber 122, so that the particles are heated by the microwaves as the particles enter the grinding chamber 122. When the particles leave the region heated by the microwave, the particles are cooled by the cryogenic gas. The cold-hot-cold cyclic change of temperature produces cracks in the particles. More than one microwave generator may be used to provide heating of the particles at different regions of the grinding chamber 122, producing multiple alternating hot and cold regions in the chamber 122. In that case, as the particles pass through the grinding chamber 122, the particles go through multiple cycles of thermal expansion and contraction, producing cracks in the particles in each cycle.

Examples of materials that crack when subjected to thermal treatment include cellulose and herbal medicines.

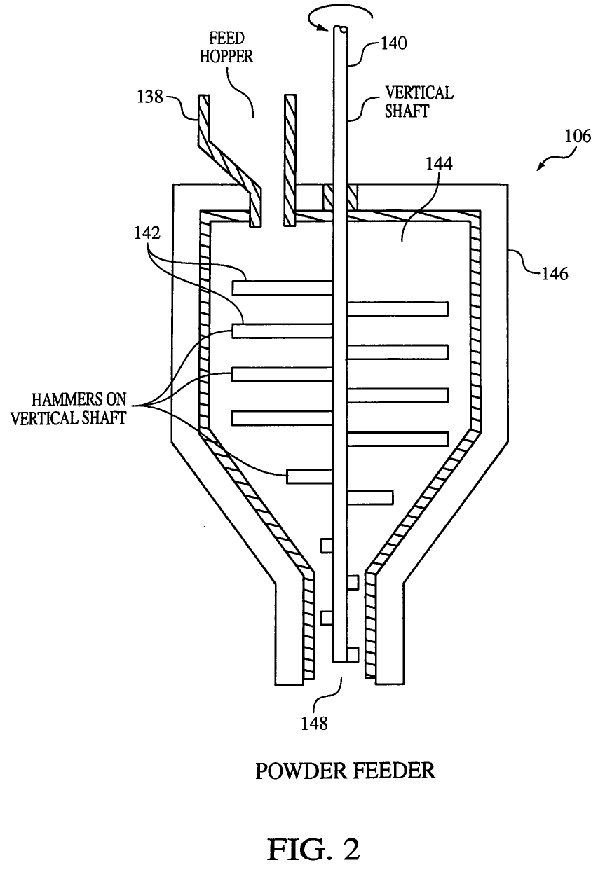

Referring to FIG. 2, the powder feeder 106 includes a receiving chamber 144 to hold solid particles before the particles are loaded into the jet mill 102. Solid particles having diameters in the range of about 100 μm to 200 μm are poured into the receiving chamber 144 through a feed hopper 138. Hammers 142 connected to a vertical rotating shaft 140 stir the solid particles to prevent the particles from aggregating. An outer jacket 146 containing liquid nitrogen surrounds the receiving chamber 144 to maintain the receiving chamber 144 at a cryogenic temperature. The particles exit the receiving chamber 144 through an opening 148 at the bottom of the powder feeder 106.

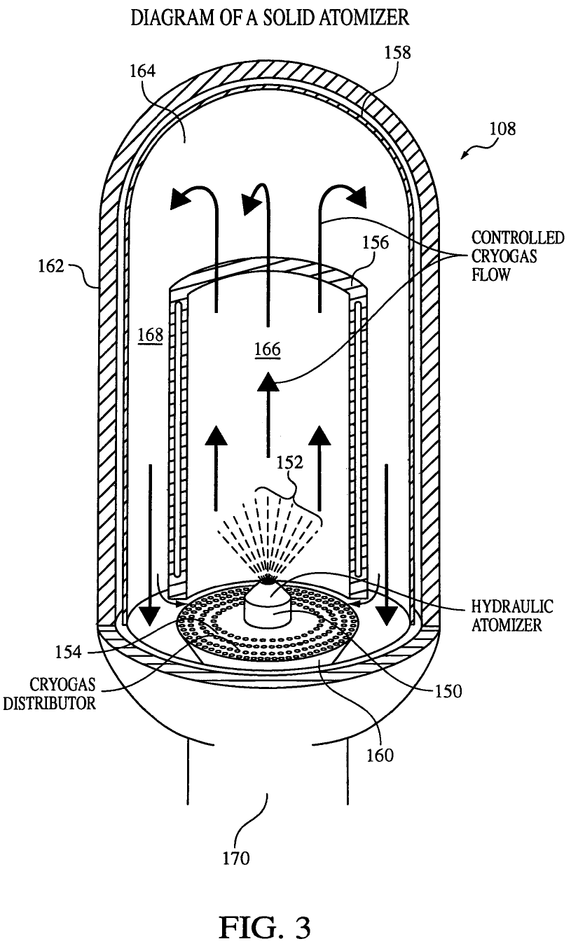

Referring to FIG. 3, a solid atomizer 108 generates solid dispersion particles 152 by passing a pressurized liquid (not shown) through a spray head 150 that is positioned in a low-pressure chamber 164. The pressurized liquid contains a material for which nano-particles are to be produced, and a dispersion medium (e.g., a solvent) that is used to disperse the material. The chamber 164 includes an inner region 166 (surrounded by a cylindrical wall 156) and an outer region 168. A cryogenic gas distributor 160 ejects cryogenic gas through nozzles 154 that are positioned beside the spray head 150. The cryogenic gas flows upward in the inner region 166 and downward in the outer region 168, reducing the temperature of the low pressure chamber 164. The cryogenic gas flows out of the solid atomizer 108 through an opening 170.

The spray head 150 is connected to a source of the pressurized liquid, which can be an emulsion (e.g., guaiacol carbonate chloroform solution in water), or a solution that contains the material dissolved in a solvent (e.g., griseomycin ethanol solution). In one example, the liquid has a high pressure of 1000 pounds-per-square-inch (psi) while inside the spray head 150. When the liquid is sprayed into the inner chamber 166, the material is finely dispersed in small droplets of the liquid at the molecular or colloidal level. Before the small droplets congregate to form larger droplets, the small droplets are cooled abruptly by the cryogenic gas and become frozen, preventing crystallization of the material. Because the material in the small droplet is frozen before it crystallizes, each of the particles 152 can contain one or more individual molecules of the material. The three dimensional structures of the molecules of the material are preserved in the solid dispersion particles 152. The solid dispersion particles 152 are drawn by the flow of the cryogenic gas from the inner region 166 to the outer region 168 and out of the solid atomizer through the opening 170.

Because the material is frozen before it crystallizes, the material in the solid dispersion particles 152 has reduced structural strength, and has a greater tendency to crack under the cryogenic condition. After the solid dispersion particles 152 are processed by the jet mills 102 and 104, and enter the cyclone collector 118, the dispersion medium (or solvent) sublimes and exits the exhaust ports 232. At least a portion of the nano-particles that are processed by the jet mills 102 and 104 will maintain the three dimensional structures of the molecules of the material. This is useful when the material contains, e.g., drugs (such as insulin), polymers, proteins, or peptides. For example, preserving the three dimensional structures of drug molecules will enhance their bioavailability.

Table 1 lists examples of materials and solvents that can form solutions suitable for generating solid dispersion particles 152 by the solid atomizer 108.

TABLE 1

| Material | Solvent |
|---|---|
| Insulin | Water |
| Taxine | Ethanol |

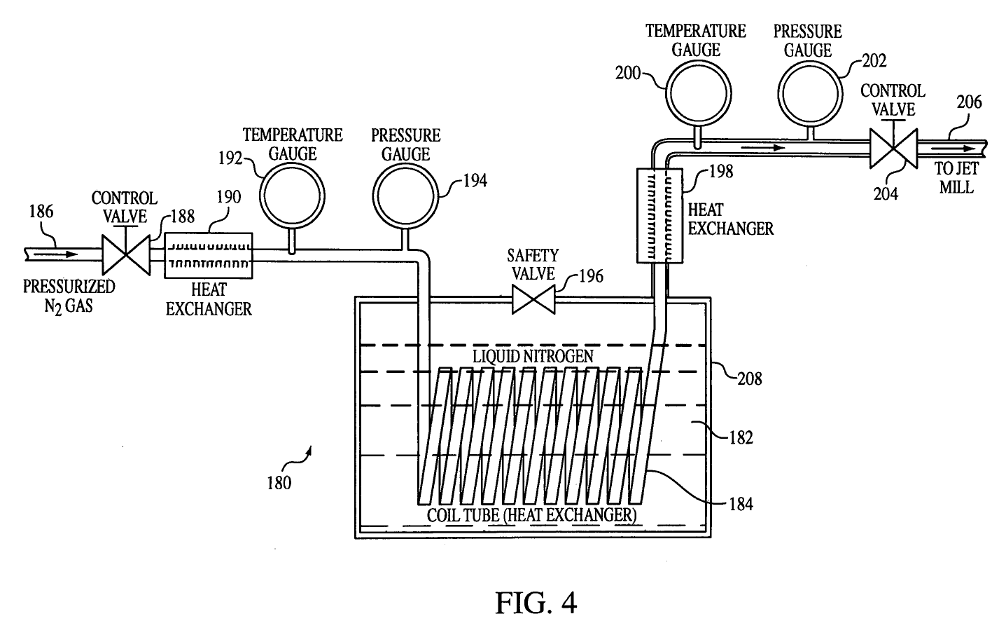

FIG. 4 shows an example of a high pressure cryogenic gas generator 180, which includes a coil tube 184 immersed in a tank of liquid nitrogen 182 maintained at about −196° C. Pressurized nitrogen gas from a nitrogen gas source (not shown) enters the cryogenic gas generator 180 through an inlet 186, passes through the coil tube 184, and is cooled to a low temperature by the liquid nitrogen 182. The amount of nitrogen gas flowing through the coil tube 184 is controlled by a control valve 188. A temperature gauge 192 and a pressure gauge 194 monitor the temperature and pressure, respectively, of the nitrogen gas before it enters the coil tube 184. The liquid nitrogen 182 is stored in a thermally insulated tank 208 that has a safety valve 196 to release nitrogen gas to prevent pressure buildup. A temperature gauge 200 and a pressure gauge 202 monitor the temperature and pressure, respectively, of the nitrogen gas leaving the coil tube 184. The pressurized cryogenic gas leaves the cryogenic gas generator 180 through an outlet 206, which is connected to the gas inlets of the jet mills 102 and 104. A control valve 204 controls the amount of nitrogen gas that enters the jet mills.

Heat exchangers 190 and 198 allow adjustment of the temperature of the pressurized cryogenic nitrogen gas. Different materials may require use of cryogenic gas at different temperatures during the jet milling process. Table 2 lists suitable temperatures for the cryogenic gas for different materials.

TABLE 2

| Material | Temperature of cryogenic gas |
|---|---|
| Griseofulvin | −140° C. |
| Albuterol Sulfate | −100° C. |
| Ibuprofene | −120° C. |
| Lecithin | −100° C. |

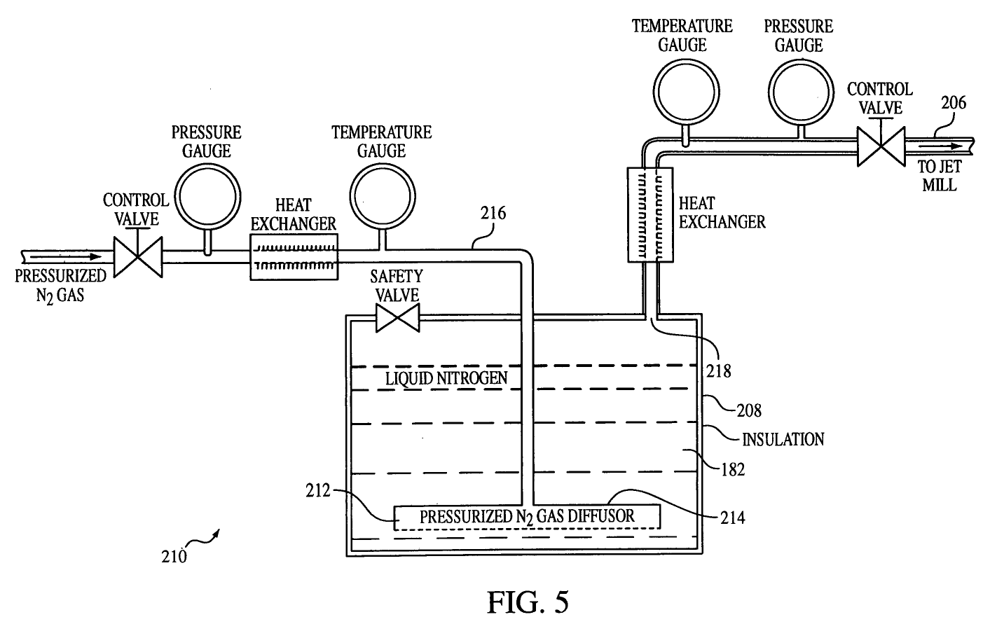

FIG. 5 shows an alternative example of a high pressure cryogenic gas generator 210, in which pressurized nitrogen gas is diffused into the liquid nitrogen 182. The pressurized nitrogen gas passes through a passage 216 that extends into the liquid nitrogen 182. The passage 216 is connected to a gas diffuser 214, which has openings to allow the pressurized nitrogen gas to enter the liquid nitrogen 182. The nitrogen gas, after being diffused into the liquid nitrogen 182, rises to the top of the liquid nitrogen 182 and exits the tank 208 through an outlet 218. The nitrogen gas that exits through outlet 218 is cooled to near the temperature of the liquid nitrogen 182, which is about −198° C.

An advantage of diffusing nitrogen gas through the liquid nitrogen 182 is that the nitrogen gas exiting through outlet 218 contains little or no water vapor. Traces of water, if any, become ice and remain in the tank of liquid nitrogen 182. This reduces the amount of moisture in the particles in the jet mills 102 and 104. Another advantage is that the nitrogen gas exiting through the outlet 218 contains little or no oxygen. Oxygen has a boiling point of −183° C., so any oxygen that passes the liquid nitrogen 182 is liquefied at the temperature of −196° C. This reduces the likelihood of oxidation of the particles in the jet mills.

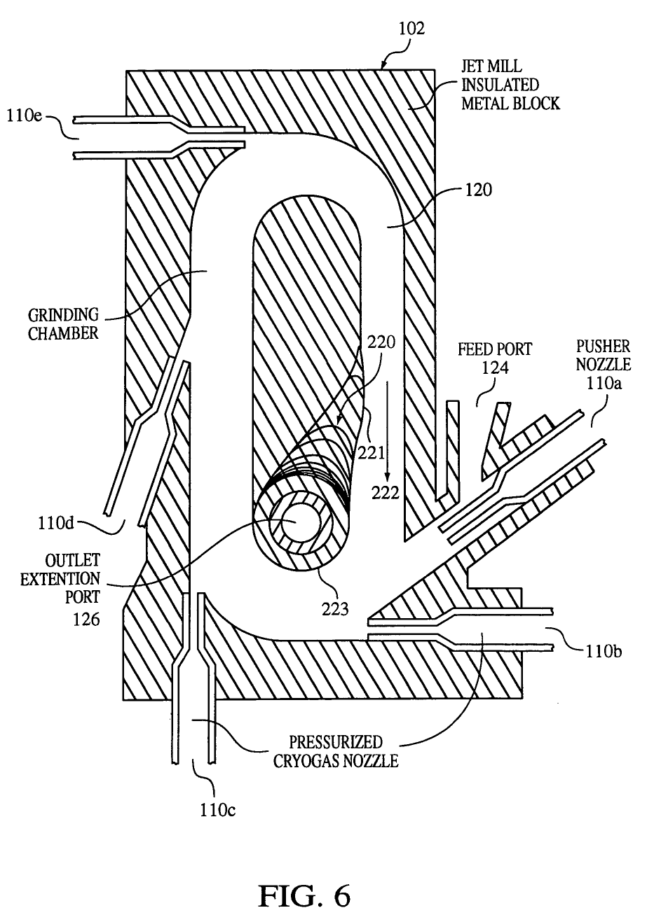

Referring to FIG. 6, the jet mill 102 includes a loop-shaped grinding chamber 120. Particles enter the chamber 120 through a feed port 124, which is coupled to the powder feeder 106 or the solid atomizer 108. Pressurized cryogenic gas is injected into the chamber 120 through nozzles 110a, 110b, 110c, 110d, and 110e to force the particles to intermix, collide with one another, and grind against the walls of the chamber 120, thereby producing smaller particles. The nozzle 110a is a pusher nozzle. Cryogenic gas rushing out of the nozzle 110a creates a suction force that pulls the particles into the feed port 124 from the powder feeder 106 or solid atomizer 108.

The cryogenic gas nozzles 110a to 110e are connected to the outlet 206 of the pressurized cryogenic gas generator 180 or 210. The nozzles are oriented so that the particles are blown in one direction (e.g., clockwise in the example shown in FIG. 6) in the grinding chamber 120. A larger portion of the nitrogen gas travels along a direction 220 and exits the chamber 120 through an outlet extension port 126. In FIG. 6, one end 221 of the outlet extension port 126 extends in a direction parallel to the plane of the figure and connects to the chamber 120. Another end 223 of the port 126 extends in a direction perpendicular to the plane of FIG. 6, and is coupled to a connector tube 128 (FIG. 1), which in turn is coupled to the feeding port 129 of the jet mill 104. The cross section of the end 223 is on a plane that is different from the plane of the cross section of the grinding chamber 120.

A smaller portion of the nitrogen gas travels along a direction 222 and loops around the chamber 120 again. The nitrogen gas carries the particles with it, so that a larger portion of the particles travel along the direction 220 and exit the chamber 120 through the outlet extension port 126. A smaller portion of the particles travel along the direction 222 and loop around the chamber again, going through another grinding cycle. The chamber 120 is insulated to maintain the nitrogen gas at a cryogenic temperature.

Jet mill 104 has a configuration similar to the jet mill 102. Cryogenic gas enters the chamber 104 through inlets 110f, 110g, 110h, 110i, and 110j (see FIG. 1). The particles are jet milled in the grinding chamber 122, similar to the jet milling process in the jet mill 102. The chamber 122 has an outlet extension port 130 that is coupled to the cyclone collector 118 through a connecting tube 240.

The particles that enter the feeding port 124 of the jet mill 102 can have dimensions in the range of about 100 μm to 200 μm, and the particles that exit the outlet extending port 130 of the jet mill 104 (referred to as the “final particles”) can have dimensions smaller than 1 μm. The dimensions of the final particles can be adjusted by varying the temperature and pressure of the gas injected into the grinding chambers of the jet mills 102 and 104. In one example, more than half of the final particles have dimensions smaller than 200 nm.

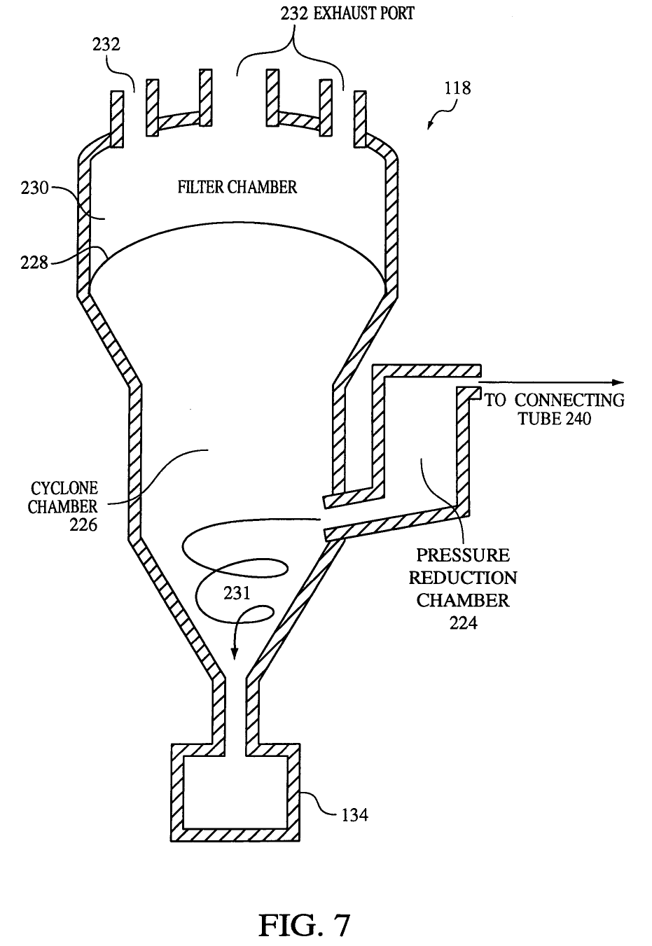

Referring to FIG. 7, the cyclone collector 118 includes a pressure reduction chamber 224 that is connected to the connecting tube 240. The pressure reduction chamber 224 allows the pressure of the nitrogen gas to be reduced prior to entering a cyclone chamber 226. The cyclone chamber 226 is kept at normal atmospheric pressure or at a low pressure, so that any water or solvent in the particles is evaporated due to the sudden decrease in pressure. The particles settle down (231) at a particle collector 134 due to gravity.

The cyclone collection 118 includes a filter chamber 230 that is separated from the cyclone chamber 226 by a filter 228. The filter chamber 230 has exhaust ports 232 that allow the nitrogen to diffuse into the atmosphere. The filter 228 prevents the nano-particles from leaving the cyclone chamber 226 through the exhaust ports 232.

Examples of materials that can be processed by the nano-particle generator 100 to produce particles having dimensions smaller than 200 nm include calcium oxalate, calcium sulfate, calcium phosphate, silicon dioxide, cellulose and herbs, insulin, taxine, griseofulvin, albuterol sulfate, ibuprofene, lecithin, plastic, vitamins, iron oxide, paclitaxcel, and so forth.

The nano-particle generator 100 is useful in many areas. For example, the nano-particle generator 100 can be used to prepare drugs for intravenous injection. Drugs are usually dissolved in solvents to form solutions that are administered to patients through intravenous injections. Some drugs can be dissolved in water for injection (WFI, purified water that is suitable for injection), while some drugs have to be dissolved in pharmaceutical solvents (such as glycerin, ethanol, propylene glycol, or a mixture of these solvents). For drugs that cannot be dissolved in water, aqueous suspensions containing the drugs can not be safely administered to a patient intravenously because the un-dissolved drug particles are too large, and may block micro vessels or cause thrombosis. Although the drugs may be dissolved in pharmaceutical solvents, injecting pharmaceutical solvents into the body often create undesirable side effects. By using the nano-particle generator 100 to process the drugs to generate particles having sizes smaller than 200 nm, aqueous suspensions containing the nano-sized drug particles can be safely administered to the patient through intravenous injection, as the drug nano-particles can smoothly pass micro vessels. This provides a new approach to pharmaceutical dosage form, reducing the need for pharmaceutical solvents in intravenous injections.

Although some examples have been discussed above, other implementations and applications are also within the scope of the following claims. For example, the nano-particle generator 100 includes two cascaded jet mills 102 and 104. For some materials, one jet mill 102 may be sufficient to produce nano-sized particles. More than two jet mills can be cascaded. The grinding chamber 120 and 122 can have different shapes. The ultrasonic wave generator 114 and the microwave generator 116 may both be coupled to the same jet mill 102 and/or 104. The ultrasonic wave generator 114 and the microwave generator 116 are optional.

The cryogenic gas that is injected into the grinding chambers 102 and 104 can be different from a nitrogen gas. For example, air or an inert gases can be used. An example of an inert gas is helium. A mixture of inert gases may be used.

The example described above uses a jet mill to process the particles under a cryogenic condition to generate nano-particles that have all three dimensions smaller than, e.g., 200 nm. A material can also be processed under a cryogenic condition to generate particles that have one dimension smaller than 200 nm, while the other two dimensions are larger than 200 nm (e.g., the particles may have a thin and flat shape). A material can also be processed under a cryogenic condition to generate particles that have two dimensions smaller than 200 nm, while the third dimensions is larger than 200 nm (e.g., the particles can have a needle shape). For example, ultrasound may be imposed on the material under a cryogenic condition so that the material crack into thin-flat shaped or needle-shaped particles based on its crystalline structure.

When a material is processed under a cryogenic condition, whether using a jet mill or another processing tool, the particle size of the final product may not be completely uniform, and may have a range of distribution. In one example, the particles processed by the nano-particle generator 100 and collected at the collector 134 may have sizes in a range between 50 nm to 300 nm, with more than 50% of the particles having sizes in a range between 100 nm to 200 nm. In another example, at least 5% of the particles processed under the cryogenic condition have dimensions less than 200 nm. The distribution of sizes of the particles can be adjusted by adjusting the temperature and pressure of the cryogenic gas injected into the grinding chambers, and/or by adjusting the power of the ultrasonic waves and microwaves imposed on the particles, and/or by increasing or decreasing the number of jet mills that are cascaded one after another.

What is claimed is:

1. A method comprising:
processing larger particles of a material to generate smaller particles having at least one dimension smaller than 200 nm, at least a portion of the processing performed under a cryogenic condition and based on at least a physical property of the material under the cryogenic condition.

2. The method of claim 1 wherein the cryogenic condition comprises a condition in which the temperature is less than or equal to −40° C.

3. The method of claim 2 wherein the cryogenic condition comprises a condition in which the temperature is less than or equal to −80° C.

4. The method of claim 3 wherein the cryogenic condition comprises a condition in which the temperature is less than or equal to −120° C.

5. The method of claim 4 wherein the cryogenic condition comprises a condition in which the temperature is less than or equal to −160° C.

6. The method of claim 1 wherein a physical property comprises a greater tendency to crack under the cryogenic condition.

7. The method of claim 1 wherein at least 5 percent of the larger particles are processed into smaller particles having at least one dimension smaller than 200 nm.

8. The method of claim 1 wherein at least 5 percent of the larger particles are processed into smaller particles having three dimensions smaller than 200 nm.

9. The method of claim 1 wherein the processing comprises milling the larger particles.

10. The method of claim 1 wherein the milling comprises using a pressurized cryogenic gas to cause the particles to grind against or collide with one another.

11. The method of claim 10 wherein the cryogenic gas comprises at least one of air, nitrogen, and inert gas.

12. The method of claim 11 wherein the inert gas comprises helium.

13. The method of claim 9 wherein the processing includes at least one treatment in addition to the milling, the additional treatment based on at least a physical property of the material under the cryogenic condition.

14. The method of claim 13 wherein the additional treatment comprises imposing ultrasonic waves to the particles.

15. The method of claim 13 wherein the additional treatment comprises imposing microwaves to the particles.

16. The method of claim 1, wherein the larger particles initially have at least one dimension larger than 100 μm.

17. The method of claim 1 wherein a physical property comprises the material forming cracks when subject to ultrasonic vibrations under the cryogenic condition.

18. The method of claim 1 wherein the processing comprises imposing ultrasonic waves on the particles.

19. The method of claim 1, wherein the material has a physical property such that the material forms cracks when the temperature of the material rises at least 20 degrees Celsius per second from a first temperature under the cryogenic condition to a second temperature.

20. The method of claim 1, wherein the processing comprises using microwaves to increase the temperature of the particles.

21. The method of claim 1 wherein the processing comprises exposing the particles alternately to a cryogenic condition and a non-cryogenic condition.

22. The method of claim 1, further comprising generating a cryogenic gas to process the particles.

23. The method of claim 22, wherein the cryogenic gas comprises at least one of air, nitrogen, and an inert gas.

24. The method of claim 23, wherein the inert gas comprises helium.

25. The method of claim 23, wherein the cryogenic gas has a pressure at least 100 psi.

26. The method of claim 1, further comprising spraying a liquid from a higher pressure area to a lower pressure area to generate the larger particles.

27. The method of claim 26, further comprising cooling the lower pressure area with cryogenic gas.

28. The method of claim 26, further comprising generating the liquid by dissolving a material in a solvent.

29. The method of claim 28, wherein the solvent comprises at least one of acetone, chloroform, alcohol, ether, petroleum ether, benzene, and water.

30. The method of claim 29, wherein the alcohol comprises at least one of methanol, ethanol, and isopropyl alcohol.

31. The method of claim 29, wherein the material comprises plastic.

32. The method of claim 1, wherein the larger sized particles comprise herbs.

33. The method of claim 1, wherein the larger sized particles comprise at least one of calcium oxalate, calcium sulfate, calcium phosphate, silicon dioxide, cellulose, insulin, taxine, griseofulvin, albuterol sulfate, ibuprofene, lecithin, plastic, vitamins, iron oxide, and paclitaxcel.

34. The method of claim 1 wherein the material is in a solid state at 20° C.

35. The apparatus of claim 1 wherein the material is in a liquid state at 20° C.

36. A method comprising generating a cryogenic gas by passing a gas through a passage that is cooled by liquid having a temperature less than or equal to −40° C.

37. The method of claim 36 wherein the liquid has a temperature less than or equal to −100° C.

38. The method of claim 36 wherein the liquid comprises liquid nitrogen.

39. The method of claim 36 wherein the passage comprises a coil tube immersed in the liquid.

40. The method of claim 39 wherein the gas comprises at least one of air, nitrogen, and an inert gas.

41. The method of claim 40 wherein the inert gas comprises helium.

42. A method comprising generating a cryogenic gas by passing a gas through liquid having a temperature less than or equal to −40° C.

43. The method of claim 42 wherein the liquid has a temperature less than or equal to −100° C.

44. The method of claim 42 wherein the liquid comprises liquid nitrogen.

45. The method of claim 44, further comprising removing oxygen in the gas by liquifying the oxygen in the liquid nitrogen.

46. The method of claim 42 wherein the gas comprises at least one of air, nitrogen, and an inert gas.

47. The method of claim 46 wherein the inert gas comprises helium.

48. A method comprising:
generating small particles having three dimensions smaller than 200 nm;
mixing the small particles with a liquid to generate a solution; and
administering the solution to a human body through an intravenous injection.

49. The method of claim 48 wherein the small particles are generated according to a process in which at least a portion of the process is performed at a temperature less than or equal to −40° C.

50. The method of claim 49 wherein the process comprises jet milling larger particles to generate the small particles.

51. The method of claim 48 wherein the small particles comprise a pharmaceutical agent.

52. A method comprising:
generating droplets of a liquid containing a material that is dispersed in a dispersion medium; and
cooling the droplets under a cryogenic condition to generate solid dispersion particles that contain the material.

53. The method of claim 52 wherein the cryogenic condition comprises a condition in which a temperature is less than or equal to −40° C.

54. The method of claim 53 wherein the cryogenic condition comprises a condition in which a temperature is less than or equal to −100° C.

55. The method of claim 52 wherein generating the droplets comprises passing the liquid from a higher pressure region through an opening to a lower pressure region.

56. The method of claim 53 wherein cooling the droplets under the cryogenic condition comprises using a cryogenic gas to cool the droplets.

57. The method of claim 52 wherein at least a portion of the solid dispersion particles each contains a single molecule of the material.

58. An apparatus comprising:
a jet mill to receive larger sized particles and a cryogenic gas to cause the larger sized particles to be milled into smaller sized particles, at least 5 percent of the smaller sized particles having three dimensions smaller than 200 nm.

59. The apparatus of claim 58, further comprising a cryogenic gas generator to generate the cryogenic gas.

60. The apparatus of claim 58 wherein the jet mill comprises an insulated chamber to maintain a temperature equal to or below −40° C.

61. The apparatus of claim 58, further comprising a collecting chamber to collect the smaller sized particles.

62. The apparatus of claim 61 wherein the collecting chamber comprises a low-pressure chamber having a pressure lower than 1 atm.

63. The apparatus of claim 58, further comprising an ultrasonic wave generator to direct ultrasonic waves toward the particles.

64. The apparatus of claim 58, further comprising a solid atomizer to generate the larger sized particles from a liquid.

65. The apparatus of claim 64 wherein the solid atomizer comprises a spray head to spray the liquid from a high pressure region to a low pressure region.

66. The apparatus of claim 65, further comprising nozzles to inject cryogenic gas around the spray head to cool liquid droplets sprayed out of the spray head.

67. The apparatus of claim 58, further comprising a heater to heat the particles while the particles are being milled.

68. The apparatus of claim 67 wherein the heater comprises a microwave generator.

69. The apparatus of claim 58 wherein the cryogenic gas comprises at least one of nitrogen, air, and an inert gas.

70. An apparatus comprising:
a particle processor to receive larger particles and to process the larger particles into smaller particles having at least one dimension smaller than 200 nm, at least a portion of the particle processor being maintained under a cryogenic condition so that at least a portion of the processing of the larger particles is performed under the cryogenic condition.

71. The apparatus of claim 70 wherein the particle processor comprises a jet mill to mill the larger particles under the cryogenic condition.

72. The apparatus of claim 70 wherein the cryogenic condition comprises a condition in which the temperature is less than or equal to −40° C.

73. The apparatus of claim 72 wherein the cryogenic condition comprises a condition in which the temperature is less than or equal to −100° C.

74. An apparatus comprising:
a container that contains a liquid maintained at a temperature less than or equal to −40° C.; and
a passage having at least a portion that is immersed in the liquid, the passage having a first opening to receive a gas having a temperature higher than −40° C., the passage having a second opening to output the gas after the gas passes the portion that is immersed in the liquid.

75. The apparatus of claim 74 wherein the liquid comprises liquid nitrogen.

76. The apparatus of claim 74, further comprising a source to generate the gas.

77. The apparatus of claim 76 wherein the gas comprises at least one of nitrogen, air, and an inert gas.

78. The apparatus of claim 74 wherein the passage comprises a coil tube.

79. An apparatus comprising:
a container that is partially filled with a liquid maintained at a temperature less than or equal to −40° C.;
a first passage having a first opening to receive a gas having a temperature higher than −40° C., the first passage having a second opening that is immersed in the liquid to output the gas into the liquid, the gas forming bubbles in the liquid that emerge from the liquid and enter a space in the container above the liquid; and
a second passage to receive the gas in the open space.

80. The apparatus of claim 74 wherein the liquid comprises liquid nitrogen.

81. The apparatus of claim 74, further comprising a source to generate the gas.

82. The apparatus of claim 76 wherein the gas comprises at least one of nitrogen, air, and an inert gas.

83. An apparatus comprising:
means for reducing a temperature of particles to less than or equal to −40° C.; and
means for processing the particles to generate smaller particles.

84. The apparatus of claim 83, wherein the means for reducing the temperature of particles comprises a cryogenic gas generator to generate a gas having a temperature that is less than or equal to −40° C.

85. The apparatus of claim 83, wherein the means for processing the particles comprises a jet mill.
